(19) United States
(12) Patent Application Publication
(10) Pub. No.: US 2012/0202193 A1
(43) Pub. Date: Aug. 9, 2012

(54) DISPOSABLE DEVICE FOR STORING BIOLOGICAL LIQUIDS AND USE THEREOF TO DETECT MATERIALS, PARTICLES, AND/OR CELLS

(76) Inventor: Hans-Werner Heinrich, Greifswald (DE)

(21) Appl. No.: 13/496,653

(22) PCT Filed: Aug. 30, 2010
(86) PCT No.: PCT/DE2010/001002

§ 371 Date: Apr. 26, 2012

(30) Foreign Application Priority Data
Sep. 18, 2009 (DE) 10 2009 041 895.4

(51) Int. Cl. G01N 1/18; B01D 35/00; C12M 1/12; C12Q 1/02; C12Q 1/70

(52) U.S. Cl. 435/5; 435/29; 436/177; 435/283.1; 422/534

(57) ABSTRACT

The invention relates to a disposable device, in which the target substances are bound to solid carriers, preferably microparticles. A mechanism according to the invention enables the solid carriers to be easily concentrated to a volume of appropriate size. A further device according to the invention makes it possible for the solid carriers in the device to be subjected to known isolating, amplifying and/or detection reactions. The device is especially suitable for the enrichment and isolation and/or detection of substances in, or extracted from, biological liquids.

The device according to the invention for obtaining, storing and processing substances extracted from liquids consists of a cylinder for holding the liquids; solid carriers; seals on both ends of cylinders which can be perforated with a hollow needle, where at least one of the seals is formed as a piston disc or can be perforated; separating membranes; hollow needles for introducing and draining liquids; and possibly a stamp for actively filling the cylinder.

The invention relates to a disposable device in which the target substances are bound to solid carriers, preferably microparticles. A mechanism according to the invention enables the solid carriers to be easily concentrated to a volume of appropriate size. A further device according to the invention makes it possible for the solid carriers in the device to be subjected to known isolating, amplifying and/or detection reactions. The device is especially suitable for the enrichment and isolation and/or detection of substances in, or extracted from, biological liquids.

Biological research; identifying diseases and pathogens,

Environmental pollution and food monitoring require simple processes for isolating and/or detecting the target substance. Research laboratories need to save working time and in diagnostics there is a need for useful ways of supplementing machine-aided processes by means of rapid tests that provide a basis for making decisions on necessary emergency medical treatment. Persons in at-risk groups, such as allergy sufferers, need simple tests to rule out the presence of allergens.

Strip tests have been used for decades as a means of rapid testing in diagnostics. They have proven to be a successful method of detecting diffusible, dissolved substances such as hormones and proteins, which must be present in the sample at least in the nanogram-range per ml. These processes do not permit the detection of lower concentrations of dissolved substances and particles and/or cells whose size prevents them from diffusing into the immunochemical reaction space.

Particles loaded with specific antibodies are successfully used for enriching dissolved substances. This enables the target substance from a large volume to be concentrated in a small reaction space. Likewise, particles and/or cells can also be specifically enriched with catcher particles.

The target substance is isolated and/or detected by means of a sequence of physical and chemical reactions such as washing with buffer, releasing the target substance, incubating with marked antibodies, washing and detecting with enzyme chemicals. All of the reagents are dissolved in liquids or are liquid themselves.

The problem to be solved was to develop a device that is suitable for aseptic sampling and storing and that can simultaneously be used as a reaction space for enriching and/or detecting target substances. It was also intended that the device should enable the process to be mechanised to a large extent.

This problem has been solved in accordance with the claims.

Thus the invention concerns a device according to claim 1, its use according to claim 15 and a process according to claim 19. The further claims concern preferred embodiments of the invention.

This invention is based on the surprising observation that a device consisting of or incorporating
a cylinder (1) for holding the liquids, which is preferably evacuated,
solid carriers (2)
at least partially elastic seals(3A; 3B) on both ends of the cylinder (1), which can be particularly penetrated by a hollow needle, where at least one of the seals (3A) is formed as a piston disc or can be perforated
separating membranes (4A, 4B)
hollow needles (5) for introducing and draining liquids
and possibly a suitable mount for fastening the device for the purpose of processing samples
and possibly a stamp (9) for actively filling the cylinder (1) with liquid, where the stamp (9) is preferably loosely connected with the piston disc (3A) by means of a predetermined breaking joint (10), and in particular is formed in such a way that, after active filling, it is broken at the predetermined breaking joint as a result of tension on the stamp (9) and the device can then be used for storing and/or processing the sample as per the invention
   is suitable for obtaining, storing and/or processing substances from liquids in a particularly simple and economical way, in particular for use as a disposable device.

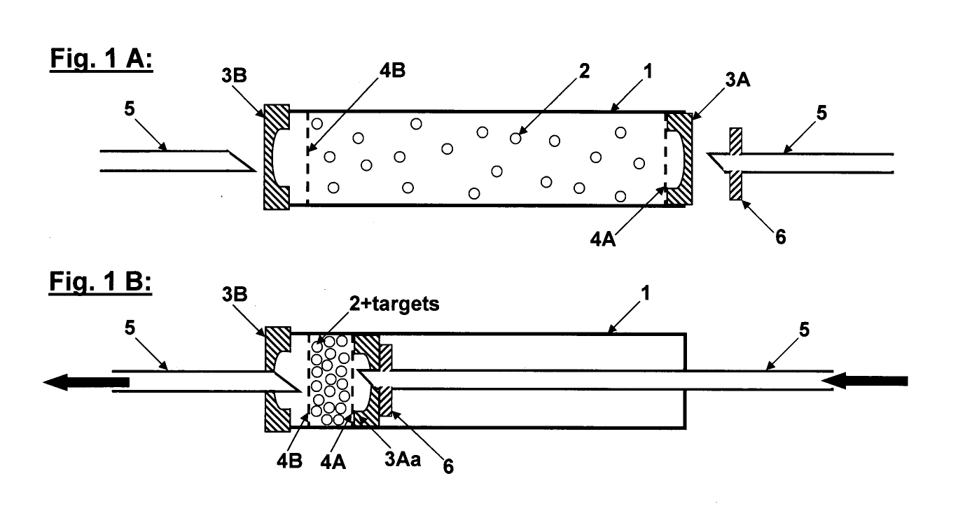

The principle underlying the invention can be seen from FIGS. 1-5, which show several possible embodiments of the invention and its utilisation.

For the at least partially elastic seals (3A; 3B), which can be particularly penetrated by a hollow needle, suitable commercially available systems should preferably be used, particularly seals which are composed of or are formed with rigid plastic (such as polypropylene) and a flexible material that seals itself after being penetrated by a hollow needle (3A1, 3B1), such as (synthetic) latex or silicone.

In addition, the seals (3A; 3B) are preferably to be formed in such a way that they permit the liquid to flow through the solid carriers in the cylinder (1) as linearly as possible.

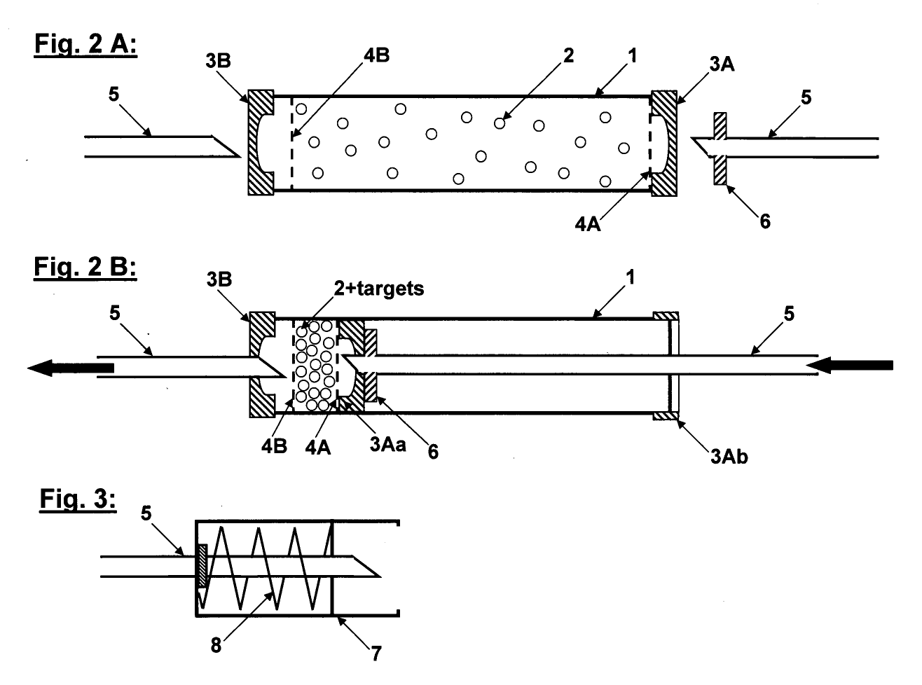

A particularly preferred design form of the invention concerns a device where
the cylinder (1) is a hollow cylinder,
at least one of the hollow needles (5) has a catch device (6) which is preferably formed as a hollow cylinder disc that is penetrated by the hollow needle (5) and in which at least one seal (3A) is a piston disc (see FIG. 1 A) or can be perforated in such a way that a piston disc (3Aa) can be removed from the seal (3A) by touching the catch device (6) (see FIGS. 2 A and B), with the piston disc preferably touching the inside wall of the cylinder (1) over its entire cross-section (see FIG. 1 B or FIG. 2 B), and
two separating membranes (4A, 4B) are arranged in the cylinder (1), between which the carriers (2) are located, with at least one separating membrane (4A) being mounted adjustably and being preferably fastened on the piston disc (3Aa), so that the carriers (2) can be moved in the direction of the other separating membrane (4B) by means of the piston disc movement (4A), the pore width of the separating membranes (4A, 4B) being, in particular, smaller than the diameter of the solid carriers used.

According to the invention, it is especially suitable if the at least one seal (3A) on the side towards the cylinder (1) has a recess over which the separating membrane (4A) is preferably stretched or fastened. This recess is preferably formed in such a way, and in particular is sufficiently deep, that the tip of the hollow needle with the catch device (5) cannot touch the separating membrane (4A) when puncturing the seal (3A) and subsequently moving the piston disc (3Aa) through the cylinder.

In an extra easy to operate embodiment of the device, the seals 3A and 3B are formed identically (and preferably the separating membranes as well) with separating membrane 4A being fastened on seal 3A and separating membrane 4B being fastened on seal 3B, and—particularly advantageously—the hollow needles (5) also each have a catch device (6) for introducing and draining liquids.

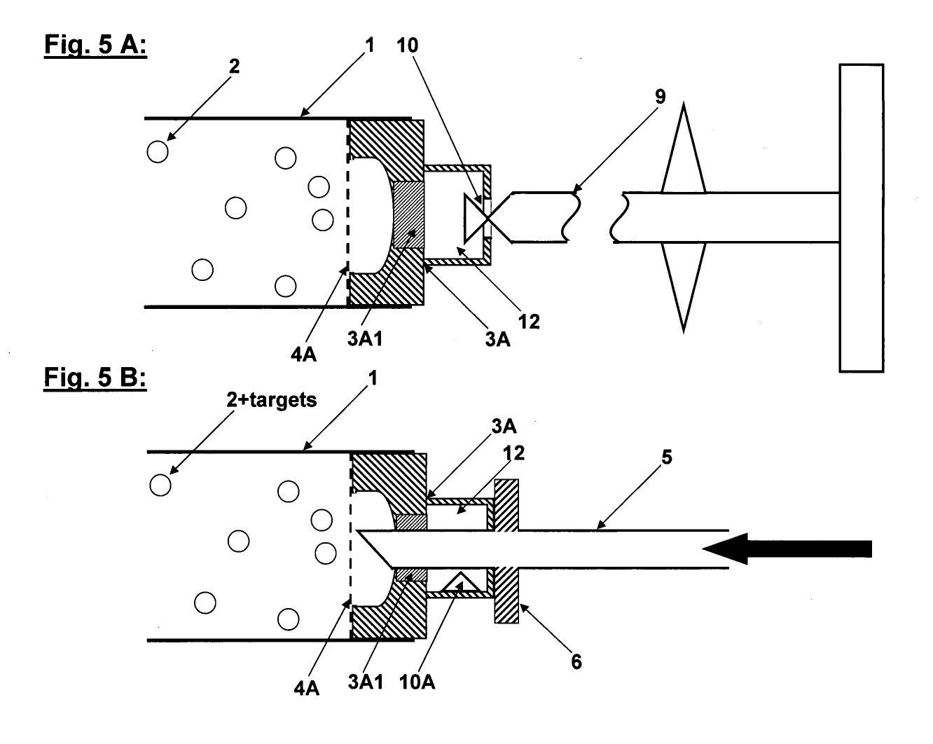

For a particularly suitable device that can be actively filled (with blood, for example) part of the stamp (9), in particular the stamp end which is used for power transmission to the piston disc (3A), preferably with at least a part of the predetermined breaking joint (10), is loosely mounted or located in a hollow space (12) or in the recess of the piston disc (3A) (refer to FIG. 5A), preferably in such a way that this part of the stamp (9) remains in the hollow space or recess (12) (10A, refer to FIG. 5B) when the stamp (9) is broken off at the predetermined breaking joint (10).

According to the invention, the solid carriers (2) can have any geometrical shape and are preferably particles (in particular microparticles), with ligands that can identify and bind to the target substance being bonded, preferably covalently or adsorptively, on the surface of the solid carriers (2).

Antibodies and/or nucleic acids are particularly suitable as ligands.

In a preferred embodiment of this device, one of the elastic seals (3A) acts as a piston disc (3Aa).

In another preferred embodiment, at least one of the hollow needles (5) has a catch device (6) which, after perforating the elastic seal, presses this into the cylinder as a piston disc.

A device according to the invention is particularly suitable, if another hollow needle penetrates into the other end of the device, preferably parallel to or simultaneously with the perforation of the piston disc.

In a further preferred embodiment of the device, at least the draining opening, e.g. the opening of the draining hollow needle (5), is fitted with a filter membrane whose pore width is smaller than the diameter of the solid carriers used.

In another preferred embodiment of the device according to the invention, the introducing and/or draining hollow needle (5) is mounted inside a sleeve with a spring mechanism; see, for example (FIG. 3).

A device according to the invention is particularly suitable where any liquids can be injected into the device through the introducing hollow needle (5) and/or where the device can be mounted on a suitable bracket that may have a mechanism for cooling and/or heating.

For a wide range of applications a device is particularly suitable, where the functionalised particles can be enriched in front of the draining end of the device to a volume that serves the desired purpose by pressing the piston disc (3Aa) into the cylinder (1).

Another aspect of the invention concerns using the device according to the invention for isolating and/or enriching and/or detecting substances extracted from or in biological liquids such as blood, liquor, secretions and/or excretions, prepared tissues, cell, yeast and bacterial cultures, rinsing samples of organs, foodstuffs and/or the environment where the substances are preferably toxins, proteins, nucleic acids, immunocomplexes, cell organelles, viruses, bacteria, protozoa, parasites, and/or somatic cells.

In principle, the device is particularly suitable for all possible carrier-bonded reactions, in particular for isolating, amplifying, detecting and/or for further modifying these substances.

The device is advantageously used in such a way that the substances are detached from the carriers (2) by known reactions such as a pH shift and can be further processed, e.g. modified and/or quantified, outside of the device.

A further aspect of the invention concerns a process for obtaining, storing and/or processing substances extracted from liquids, including the steps of:
providing a device as per the invention,
filling said device with a liquid, such as blood, preferably containing at least one target substance, preferably—for example with an evacuated cylinder (1)—by the seal (3B or A) being perforated by the hollow needle located outside of the punctured vein, using a commercially available device for filling blood collection tubes (Vakutainer®). The device is withdrawn after filling from the hollow needle. The perforation is sealed by the elastic material, preventing the sample from leaking out. The device can be stored and/or transported when full.
binding of the at least one target substance to catcher structures which are covalently or adsorptively bound to the surface of the solid carriers (2), preferably with a waiting period of 10-60 minutes
for processing the sample, a hollow needle (5) with a catch device (6) is used to perforate seal 3A
perforating the other seal (3B) with another hollow needle (5) and reducing the reaction space by pressing the catch device (6), which is preferably a disc firmly connected with the hollow needle (5), against the seal (3A) so that if the seal (3A) is formed as a piston disc (3Aa), the seal (3A) is pressed into the cylinder (1),
   or, if the seal (3A) can be perforated, a piston disc (3Aa) is detached from, preferably stamped out of, the seal (3A), and the detached piston disc (3Aa)) is pressed into the cylinder (1), with the separating membrane (4A), which is preferably fastened on the piston disc (3Aa), being moved in the direction of the other separating membrane (4B) and with the liquid, which is preferably depleted by the at least one target substance, being drained out of the cylinder via the separating membrane (4B) and the other hollow needle (5).
processing the target substance bonded on the solid carrier by introducing into the reaction space liquids which are appropriate to the method via the introducing hollow needle, and possibly
detaching the at least one target substance bonded to the carriers (2) and draining the detached target substance out of the cylinder (1), preferably by conducting a medium, such as a salt solution, an acid or alkaline solution, through one of the hollow needles (5) and into the reduced reaction space, and draining the medium with the detached target substance out of the cylinder through the other hollow needle (5).

For a preferred active process, the device as per the invention is filled with a liquid, such as blood, through the at least partially elastic seal 3B being perforated at the elastic point (3B1) using customary hollow needle systems (11), with the other end of the hollow needle (11) extending into a sample-taking space, such as a blood vessel, and the liquid, such as blood, being transported through the hollow needle (11) from the sample-taking space into the device by means of the stamp (1) being pulled out of the cylinder (1) and the stamp (9) being broken at the predetermined breaking joint (10) after the device has been filled with the liquid in such a way that the piston disc (3A) is prepared for being perforated by the hollow needle (5) with the catch device (6).

This process is particularly suited to the aforementioned uses of the device as per the invention.

Other advantageous properties and features of the invention are evident from the (non-exhaustive) examples given below of disposable devices and their use as per the invention, with FIGS. 1-5 showing various design forms of the invention.

FIGS. 1 A and 1 B show one variation of the device as per the invention where the seal (3A) is formed as a piston disc (3Aa).

FIGS. 2 A and 2 B show one variation of the device as per the invention where the seal (3A) is formed as a seal (3A) that can be perforated, from which a piston disc (3A) can be detached by means of the catch device (6), so that a piston disc (3Aa) and the seal part remaining on the cylinder opening (3Ab) are formed from the seal (3A).

FIG. 3 shows the advantageous mounting of a hollow needle (5) in a safety sleeve (7) with a spring mechanism.

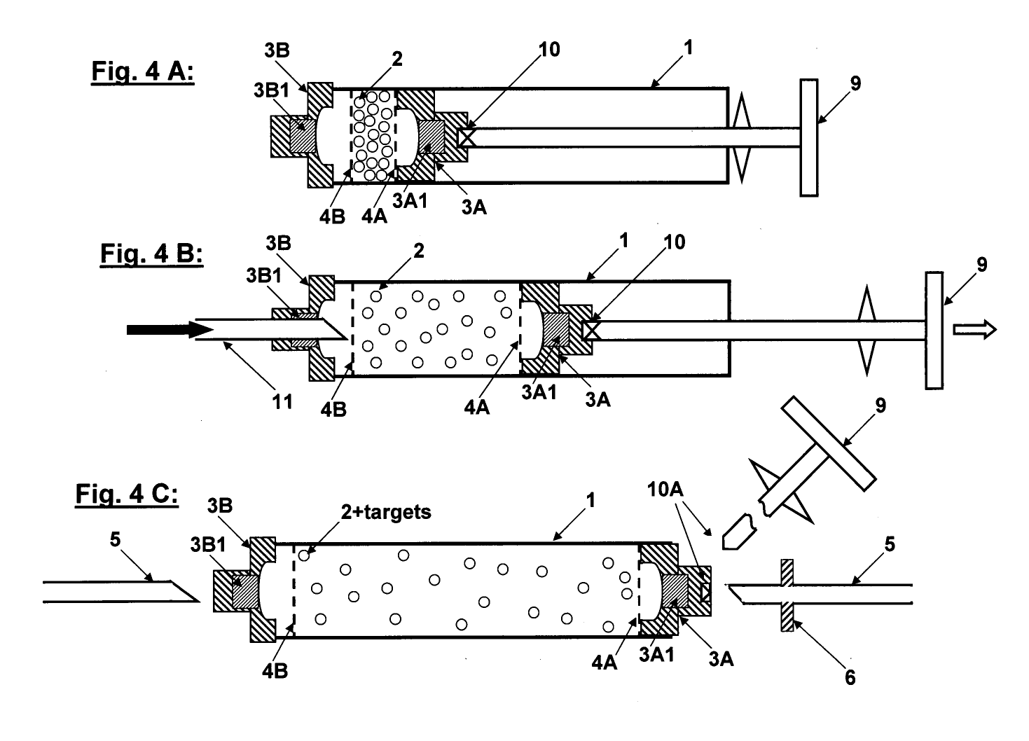

FIG. 4 shows a syringe principle as per the invention for filling the cylinder (1) with a sample (see FIG. 4B), such as blood, which is then subsequently further processed, in particular as per FIG. 1-3.

FIG. 5 shows a design form of the syringe system as per the invention (an excerpt) for actively filling the device, with FIG. 5A showing the device after removing the stamp (9) while simultaneously filling the device with a sample, such as blood, and with the stamp (9) subsequently being broken off at the predetermined breaking joint (10) in order for the hollow needle (5) with the catch device (6) to then penetrate the elastic part (3A1) of the seal (3A), which is formed as a piston disc (FIG. 5B), as a result of which the reaction space is reduced by shifting the piston disc (3A) and pushing separating membrane 4A into the cylinder (1) as per FIG. 1B.

The disposable device is based on a tube that is sealed on both sides with an elastic material so as to be air-tight. Both sides of the tube can be used temporarily for introducing and draining liquids by means of a hollow needle such as a cannula. The disposable device can be under negative pressure, which makes it possible to fill it passively. Inside the disposable device are solid carriers, preferably microparticles, to which the target substances are bonded. The target substances are identified and bonded by means of catcher structures which are able to identify the target substance sufficiently precisely and then bond it to themselves. These structures are oligo- or polypeptides, preferably antibodies with a known specificity or nucleic acids with a known nucleotide sequence. The catcher structures are bonded covalently or adsorptively to the solid carriers by means of known processes. When agglutinating sample material, such as blood, is used, a sufficient quantity of an anti-agglutinating substance, such as heparin, EDTA or citrate should be introduced into the disposable device.

After the disposable device is filled, for example with blood, the target substances are bonded to the solid carrier by means of the catcher structures. This process is generally completed after 10-60 minutes.

The solid carriers act as a support for the subsequent processing of the sample.

The reaction space can now be reduced as per the invention. This is done by using a disc firmly connected with a hollow needle and a possible stamping mechanism to press one end of the elastic seal into the disposable device like the piston disc of a syringe. At the same time as the piston disc is perforated, the other end of the device is perforated with a hollow needle. Preferably, the device should be in a vertical position so that any trapped air can be expelled through the flow-out hollow needle. Inside the disposable device, in front of the draining hollow needle, is a separating membrane with a pore diameter that lets the sample material pass, but not the solid carriers. For blood, a diameter of 20-30 μm has proven suitable. As an option, a membrane of the same type can be mounted in front of the piston disc. By moving the piston disc in the direction of the flow-out, the microparticles can be enriched to the desired density between the piston disc and the separating membrane. In this compartment, which is defined as the “reaction space” as per the invention, all types of carrier-bonded reactions can be carried out, and these can easily be adapted to the system by skilled technicians. The required liquids are delivered in the desired sequence and with the desired dwelling time through the introducing hollow needle. Draining is conducted passively through the flow-out hollow needle. The reaction space can be cooled and heated by means of known processes. The target substance can be made visible and semiquantified by means of an immunochemical reaction or the target substance can be removed from the reaction space for further processing by means of a suitable liquid.

For active filling of the device, it is particularly appropriate to utilise a syringe with an additional piston stamp (9) that is loosely connected with piston disc 3A and has a predetermined breaking joint (10). The internal part of the piston stamp remains in the hollow space of the piston disc after removing the piston stamp. The piston disc has an elastic and self-sealing element which can be penetrated with a hollow needle (3A1), as set out in the system described above. Before the taking of a sample, the device is filled with the desired carriers (2) and other substances required for the respective application, e.g. anti-coagulation agents. When filling the device, the at least partially elastic seal 3B is penetrated at the elastic point (3B1) by known hollow needle systems (11). The other end of the hollow needle is in the sample-taking space, such as a blood vessel. The sample is transferred into the device by pulling on the stamp (9). When stamp 3A reaches the end of the device, the predetermined breaking joint of the stamp is exposed. The hollow needle is withdrawn from 3B and the stamp (9) is broken off. The device can now be used in the same way as the aforementioned passively filled device. The fragment of the piston stamp remaining in the piston disc is pressed to the side by the inserted hollow needle (5).

The hollow needles (5) are preferably shaped as safety needles. The needles are therefore encased in a sleeve (7). Once the sleeve reaches the end of the device, further forward pressure causes the hollow needle to be introduced into the device. The hollow needle has a spring mechanism (8) which brings the sleeve back into its former position when the needle is pulled back.

The use of a motor control unit makes this device attractive for biological research, where specific processes, such as rinsing, separating by means of centrifuging and pipetting solutions, have to be done manually despite the availability of user-friendly kits.

Application of the invention is particularly preferred for research involving the isolation of cells, proteins and/or nucleic acids.

Due to its inexpensive components and easy operability, the invention is particularly suitable for rapid tests, wherein:
rapid tests for diagnosing diseases via substances that have hitherto had to be enriched
rapid tests for directly detecting pathogens in body fluids
rapid tests for detecting tumour cells in blood, secretions and excretions
rapid tests for determining cellular immune status
rapid tests for detecting allergens in food and the environment, and
rapid tests for averting dangers (such as bioterrorism)
   being able to be conducted especially simply and cheaply

The invention also concerns a kit for obtaining, storing and processing substances extracted from liquids, which consists of or incorporates the component parts (1)-(6), possibly the component parts (9)-(10) and preferably component parts (7) and (8) in accordance with claims 1-14, which makes it possible to set up and utilise the device as per the invention.

### LEGEND FOR THE FIGURES

(1) cylinder for holding the liquids,

(2) solid carriers

(3) seals (3A; 3B) which are at least partially elastic (3A1; 3B1), on both ends of the cylinders (1)

(3Aa) piston disc

(3Ab) the part of the seal remaining on the end of cylinder following perforation (3A)

(4) separating membranes (4A, 4B)

(5) hollow needles for introducing and draining liquids

(6) catch device

(7) safety sleeve

(8) spring

(9) stamp

(10) predetermined breaking joint

(10A) predetermined breaking joint(s) after breaking

(11) commercially available cannula, e.g. for blood collection

(12) hollow space or recess in the piston disc (3A) which is at least partially enclosed by the piston disc (3A)

In FIGS. 1B, 2B, 4B and 5B, the introducing and draining of liquids through the device is indicated by a black arrow (showing the direction of flow).

Another arrow in FIG. 4B shows the movement of the stamp (9) with piston disc (3A).

1-22. (canceled)

23. A device for obtaining, storing and processing substances extracted from liquids, consisting of or incorporating
a cylinder (1) for holding liquids,
solid carriers (2),
at least partially elastic seals(3A; 3B) on both ends of the cylinder (1), which can be particularly penetrated by a hollow needle, where at least one of the seals (3A) is formed as a piston disc or can be perforated
separating membranes (4)
hollow needles (5) for introducing and draining liquids
possibly a suitable mount for fastening the device for the purpose of processing samples,
and possibly a stamp (9) for actively filling the cylinder (1) with liquid, with the stamp (9) preferably being loosely connected with the piston disc (SA) by means of a predetermined breaking joint (10) and, in particular, being formed in such a way that, after active filling, it breaks off at the predetermined breaking joint as a result of tension on the stamp (9); the device can now be used for storing and/or processing the sample as per the invention.

24. A device according claim 23, characterised thereby
the cylinder (1) is a hollow cylinder,
at least one of the hollow needles (5) has a catch device (6) which is preferably formed as a hollow cylinder disc that is penetrated by the hollow needle (5) and in which at least one seal (3A) is a piston disc or can be perforated in such a way that a piston disc (3Aa) can be removed from the seal (3A) by touching the catch device (6), with the piston disc preferably touching the inside wall of the cylinder (1) over its entire cross-section,
the at least one seal (3A) on the side towards the cylinder (1) has a recess over which the separating membrane (4A) is preferably stretched, with the recess preferably being formed in such a way that the tip of the hollow needle with the catch device (5) cannot touch the separating membrane (4A) when puncturing the seal (3A) and subsequently moving the piston disc (3Aa) through the cylinder, and/or
two separating membranes (4A, 4B) are arranged in the cylinder (1), between which the carriers (2) are located, with at least one separating membrane (4A) being mounted adjustably and being preferably fastened on the piston disc (3Aa), so that the carriers (2) can be moved in the direction of the other separating membrane (4B) by means of the separating membrane (4A).

25. A device according to claim 23, characterised thereby that the cylinder is preferably evacuated or that a part of the stamp (9), in particular the stamp end is loosely mounted in a hollow space (12) or recess of the piston disc (3A), preferably in such a way that when the stamp (9) is broken off at the predetermined breaking joint (10) at least part of the stamp (9) remains in the hollow space or recess (12).

26. A device according to claim 23, characterised thereby that the solid carriers can have any geometrical shape, but preferably particles, in particular microparticles, are used.

27. A device according to claim 26, characterised thereby that ligands which can identify and bind to the target substance are covalently or adsorptively bonded to the surface of the solid carriers.

28. A device according to one of the claim 26, characterised thereby that the ligands are preferably antibodies or nucleic acids.

29. A device according to claim 23, characterised thereby that one of the elastic seals acts as a piston disc.

30. A device according to claim 23, characterised thereby that a hollow needle (5) has a catch device (6) which, after perforating the elastic seal, presses this into the cylinder as a piston disc.

31. A device according to claim 23, characterised thereby that a hollow needle penetrates the other end of the device when the piston disc is perforated.

32. A device according to claim 23, characterised thereby that at least the draining opening has a filter membrane whose pore width is smaller than the diameter of the solid carriers used.

33. A device according to claim 23, characterised thereby that the introducing and draining hollow needles are mounted inside a sleeve with a spring mechanism.

34. A device according to claim 23, characterised thereby that any liquids can be injected into the device through the introducing hollow needle (5).

35. A device according to claim 23, characterised thereby that the device can be mounted on a suitable bracket, which may also provide the possibility of cooling and/or heating.

36. A device according to claim 23, characterised thereby that functionalised particles can be enriched in front of the draining end of the device to a volume that serves the desired purpose by pressing the piston disc (3Aa) into the cylinder (1).

37. Use of the device according to claim 23, for isolating and/or enriching and/or detecting materials extracted from or in biological liquids such as blood, liquor, secretions and/or excretions, prepared tissues, cell, yeast and bacterial cultures, rinsing samples of organs, foodstuffs and/or the environment, wherein these substances are preferably toxins, proteins, nucleic acids, immunocomplexes, cell organelles, viruses, bacteria, protozoa, parasites and/or somatic cells.

38. Use according to claim 37, characterised thereby that, in principle, all carrier-bonded reactions for isolating, amplifying, detecting and/or further modifying the substances are possible.

39. Use according to claim 37, characterised thereby that the substances released by means of known reactions can be further processed outside of the device.

40. A process for obtaining, storing and processing substances extracted from liquids, including the steps of
filling the device, according to claim 23, with liquid, such as blood, containing at least one target substance, preferably with the seal (3B) being perforated using customary blood collection devices for Vakutainer® and with the preferred evacuated device filling up with the sample until the pressure is equalised.
bonding of the at least one target substance to catcher structures which are covalently or adsorptively bonded to the surface of the solid carriers (2), preferably with a waiting period of 10-60 minutes
perforating the seal (3A) with a hollow needle (5) with a catch device (6) and
perforating the other seal (3B) with another hollow needle (5) and reducing the reaction space by pressing the catch device (6), which is preferably a disc firmly connected with the hollow needle (5), against the seal (3A) so that
if the seal (3A) is formed as a piston disc (3Aa), the seal (3A) is pressed into the cylinder, or, if the seal (3A) can be perforated, a piston disc (3Aa) is detached from, preferably stamped out of, the seal (3A), and the detached piston disc (3Aa)) is pressed into the cylinder (1), with the separating membrane (4A), which is preferably fastened on the piston disc (3Aa), being moved in the direction of the other separating membrane (4B) and with the liquid, which is preferably depleted by the at least one target substance, being drained out of the cylinder via the separating membrane (4B) and the other hollow needle (5).
processing or detecting the target substance bound on the solid carrier by introducing into the reaction space liquids which are appropriate to the method via the introducing hollow needle, and possibly
detaching the at least one target substance bonded to the carriers (2) and draining the detached target substance out of the cylinder (1), preferably by conducting a medium, such as a salt solution, an acid or alkaline solution, through one of the hollow needles (5) and into the reduced reaction space, and draining the medium with the detached target substance out of the cylinder through the other hollow needle (5).

41. Process according to claim 40, wherein the device is actively filled with a liquid, such as blood, through the at least partially elastic seal 3B being perforated at the elastic point (3B1) by customary hollow needle systems (11), with the other end of the hollow needle (11) extending into a sample-taking space, such as a blood vessel, and the liquid, such as blood, being transported through the hollow needle (11) from the sample-taking space into the device by means of the stamp (1) being pulled out of the cylinder (1), and, after the device has been filled with the liquid, the stamp (9) being broken at the predetermined breaking joint (10) in such a way that the piston disc (3A) is prepared for being perforated by the hollow needle (5) with the catch device (6).

42. Kit for obtaining, storing and processing substances extracted from liquids consisting of or incorporating the component parts (1) to (6), possibly the component parts (9)-(10) and preferably the component parts (7) and (8) in accordance with claim 1.
